(19) United States
(12) Patent Application Publication
(10) Pub. No.: US 2006/0057091 A1
(43) Pub. Date: Mar. 16, 2006

(54) Semitransparent cosmetics

(75) Inventors: Makoto Fujii, Tokyo (JP); Yoshikazu Konno, Tokyo (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: KOSE CORPORATION, Tokyo (JP)

(21) Appl. No.: 10/534,986

(22) PCT Filed: Nov. 10, 2003
(86) PCT No.: PCT/JP03/14249

§ 371 Date: May 16, 2005

(30) Foreign Application Priority Data
Nov. 15, 2002 (JP) 2002-331789

(51) Int. Cl. A61K 8/73

(52) U.S. Cl. 424/70.13

(57) ABSTRACT

A semitransparent cosmetic consisting of an O/W emulsion which comprises (a) a ceramide, (b) an oil component, (c) a nonionic surfactant, and (d) water and has a mean particle diameter of 100 to 300 nm; a semitransparent cosmetic as described above wherein the component (c) is polyoxyethylene hydrogenated castor oil; and a semitransparent cosmetic as described above wherein the mass ratio of the total amount of the components (a) and (b) to the amount of the component (c) lies between 3:1 and 1.2:1. The ceramide-containing O/W emulsion cosmetics are excellent in long-term stability and have beautiful appearances and a sense of luxuriousness.

### TECHNICAL FIELD

The present invention relates to a semitransparent (translucent) cosmetic, and in particular, to a ceramide-containing O/W emulsion semitransparent cosmetic excelling in long-term stability, possessing a beautiful appearance, and imparting a sense of luxuriousness.

### BACKGROUND ART

Ceramide, which is present in the horny layer of the skin, forms a lipid barrier necessary for retaining moisture and plays an important role in the maintaining of moisture in the skin. Ceramide in the horny layer is produced by the breakdown of cerebroside by an enzyme known as cerebrosidase. The ceramide is partially transformed into phytosphingosine and sphingosine by an enzyme known as ceramidase. Phytosphingosine and sphingosine play an important role in the control of cell growth and differentiation. Six different types of ceramides possessing different functions are present in the human skin.

However, since ceramides are highly crystalline, have a low solubility in other oil components, and produce crystals at a low temperature, ensuring long-term stability of cosmetics comprising a ceramide is difficult.

In order to ensure stability when using a ceramide, the use of a lipid dispersion composition comprising a phospholipid and a polyhydric alcohol has been disclosed (e.g. Japanese Patent Application Laid-open No. 11-130651).

Furthermore, as a technology for ensuring transparent solubilization of ceramide to incorporate the ceramide into a cosmetic composition in a stable manner, the use of a specific fatty acid and specific surfactant has been disclosed (e.g. Japanese Patent Application Laid-open Nos. 2001-139796 and 2001-316217). However, in order to ensure transparent solubilization of ceramide, a large amount of a surfactant is required, which impairs safety and feeling during use. If the amount of surfactant is decreased in order to obtain an excellent feeling during use, the ceramide does not solubilize transparently, resulting in a cloudy or semitransparent emulsion in many cases. In these cases, separation and creaming occur over time and it is difficult to obtain adequate long-term stability.

Therefore, an object of the present invention is to provide a ceramide-containing O/W emulsion semitransparent cosmetic which, in spite of the addition of a reduced amount of surfactant, excels in long-term stability, has a beautiful appearance, and imparts a sense of luxuriousness.

### DISCLOSURE OF THE INVENTION

After diligent research to overcome the above problems, the present inventors discovered that an O/W emulsion semitransparent cosmetic comprising at least ceramide, an oil component, a nonionic surfactant, and water with a mean particle diameter of 100-300 nm excels in long-term stability, thereby completing the present invention.

Specifically, the present invention provides the following.

An O/W emulsion semitransparent cosmetic comprising the following components (a)-(d): (a) a ceramide, (b) an oil component, (c) a nonionic surfactant, and (d) water, with a mean particle diameter of 100-300 nm.

The above semitransparent cosmetic, wherein the nonionic surfactant (c) is a polyoxyethylene hydrogenated castor oil.

The above semitransparent cosmetic, wherein the mass ratio of the total amount of the ceramide (a) and the oil component (b) to the amount of the nonionic surfactant (c) is 1.2:1 to 3:1.

The above semitransparent cosmetic, wherein the oil component (b) comprises a sterol.

The above semitransparent cosmetic, wherein the oil component (b) comprises an isostearic acid.

The above semitransparent cosmetic, wherein the nonionic surfactant (c) is polyoxyethylene (60) hydrogenated castor oil.

### BEST MODE FOR CARRYING OUT THE INVENTION

The present invention will now be described in detail.

There are no specific limitations to the ceramide used as the component (a) in the present invention as long as it is a ceramide commonly used in cosmetics. Specific examples of the ceramide include ceramide obtained from yeast, pseudo-ceramide obtained by chemical synthesis, and ceramide obtained from plants, for example, ceramides 1-6. These ceramides may be used either individually or in combination of two or more as appropriate. Of these, ceramide 2 and ceramide 3 are preferable in view of excellent long-term stability.

The oil component (b) of the present invention is an essential component necessary to ensure production of the O/W emulsion with a mean particle diameter of 100-300 nm and the semitransparent cosmetic having a beautiful appearance and imparting a sense of luxuriousness. There are no specific limitations to the component (b) as long as it is a component commonly used in cosmetics. Specific examples of the component (b) include hydrocarbons, oils and fats, waxes, hydrogenated oils, ester oils, fatty acids, higher alcohols, sterols, silicone oils, fluorine-containing oils, lanolin derivatives, oil-soluble vitamins, and vegetable oils.

Of these, sterols such as phytosterol containing cholesterol, campesterol, sitosterol, and stigmasterol are preferable in order to obtain an excellent emulsion stability increasing effect.

In order to increase solubility and long-term stability of the component (a), the component (b) further comprises a fatty acid such as stearic acid, lauric acid, myristic acid, behenic acid, isostearic acid, and oleic acid. Of these fatty acids, isostearic acid is particularly preferable. Isostearic acids originating from dimer acid, a Garbett reaction, or an Aldol condensation reaction may be suitably used in the present invention.

The nonionic surfactant used as the component (c) in the present invention is an essential component for ensuring excellent emulsion stability. There are no specific limitations to the component (c) as long as it is a nonionic surfactant commonly used in cosmetics. Specific examples of the component (c) include a polyoxyethylene glycerine fatty acid ester, polyglyceryl fatty acid ester, polyoxyethylene sorbitan fatty acid ester, polyoxyethylene hydrogenated castor oil, polyoxyethylene cholesterylether, polyoxyethylene phytosterylether, polyoxyethylene alkyl ether, polyoxyethylene polyoxypropylene alkylether, polyoxyethylene alkylphenylether, and alkylglucoside. This nonionic surfactant may be used either individually or in combination of two or more as appropriate.

Of these, polyoxyethylene hydrogenated castor oils such as polyoxyethylene (20) hydrogenated castor oil, polyoxyethylene (40) hydrogenated castor oil, polyoxyethylene (60) hydrogenated castor oil, and polyoxyethylene (80) hydrogenated castor oil are preferable in order to obtain good long-term stability, with polyoxyethylene (60) hydrogenated castor oil being particularly preferable.

Water used as the component (d) in the present invention is an essential component for preparing the O/W emulsion semitransparent cosmetic.

The semitransparent cosmetic of the present invention obtained from the above components must have an emulsion mean particle diameter of 100-300 nm. If the mean particle diameter of the emulsion is less than 100 nm, coalescence and the like of the emulsion may occur over time, and if the mean particle diameter exceeds 300 nm, creaming may occur. In either case, suitable long-term stability cannot be maintained. In the present invention, only an emulsion having a mean particle diameter of 100-300 nm can ensure excellent stability over time in the O/W emulsion comprising the ceramide (a) and the oil component (b).

In order to obtain more excellent long-term stability, the emulsion particularly preferably has a mean particle diameter of 150-250 nm.

Limiting the mean particle diameter of the emulsion within the range of 100-300 nm can prevent the cosmetic from becoming a transparent like solubilization type cosmetic and from becoming cloudy like milky lotions comprising a large amount of an oil component, and can ensure production of a semitransparent cosmetic imparting a sense of luxuriousness. In the present invention, “semitransparent” refers to light transmissivity in a range of 2-45%, wherein the light transmissivity is measured by a UV-2500PC UV-VIS recording spectrophotometer (manufactured by Shimadzu Corporation) using a 1 cm×1 cm quartz cell and light at a wavelength of 600 nm.

There are no specific limitations to the total amount of the component (a) and the component (b) used in the present invention. In order to ensure excellent long-term stability, the total amount of the component (a) and the component (b) is preferably 0.01-10 wt % (hereinafter abbreviated as “%”) and particularly preferably 0.05-5% of the total amount of the semitransparent cosmetic.

The component (c) is preferably used in an amount of 0.004-4% and particularly preferably 0.02-0.2% of the total amount of the semitransparent cosmetic.

In order to ensure excellent long-term stability, the mass ratio of the total amount of the components (a) and (b) to the amount of the component (c) is preferably 1.2:1 to 3:1, and particularly preferably 1.5:1 to 2.5:1. Conventionally, surfactants are used in an amount greater than the total amount of ceramide and oil component. However, in the present invention, the amount is appropriately selected within the above range in order to provide a semitransparent cosmetic with excellent long-term stability and imparting a sense of luxuriousness without degrading feeling during use.

In addition to the above essential components, surfactants other than the nonionic surfactants, gelling agents, water soluble polymers (obtained from animals, plants, or microorganisms, or by synthesis), antioxidants, pH regulators, perfumes, antibacterial agents, antiseptics, refrigerants, humectants, antiinflammation agents, whitening agents, cell activators, dry and rough skin improvers, blood circulation promoters, skin astringents, UV absorbers, and the like may be used in the semitransparent cosmetics of the present invention within a range that does not impair the effect of the present invention.

There are no specific limitations to the method of manufacturing the semitransparent cosmetic of the present invention. The semitransparent cosmetic can be manufactured by a method, when necessary, comprising steps such as heating and cooling, using an apparatus having ordinary emulsification and stirring capabilities. As a specific example, a method of homogeneously mixing the components (a), (b), and (c) with heating, adding the component (d), and emulsifying the mixture can be given.

A mean particle diameter in the range of 100-300 nm can be easily attained by appropriately selecting the stirring rate, stirring time, stirring temperature, and the mass ratio of the total amount of the components (a) and (b) to the amount of the component (c) within the above ranges.

As an example of a particularly preferable method for manufacturing the cosmetic of the present invention, a method of dissolving the components (a), (b), and (c) in a polyhydric alcohol such as 1,3-butyleneglycol while stirring at 90° C. and adding this solution to water heated to 80° C. can be given.

The semitransparent cosmetic of the present invention obtained in the above manner can be used in skin care cosmetics such as a face lotion, essence, massage lotion, pack, hand gel, and body gel, foundation for cosmetics, and the like. The cosmetic can be applied by hand or using a nonwoven fabric impregnated with the cosmetic.

Since the semitransparent cosmetic of the present invention has an O/W emulsion mean particle diameter in the range of 100-300 nm, the cosmetic is semitransparent, possesses a beautiful appearance, and imparts a sense of luxuriousness.

Furthermore, if polyoxyethylene hydrogenated castor oil is used as the component (c) or if the components (a), (b), and (c) are used in specific amounts, the semitransparent cosmetic of the present invention possessing excellent long-term stability in addition to the above beautiful appearance can be obtained.

### EXAMPLES

The present invention will be described in more detail by way of Examples which should not be construed as limiting the present invention. In the examples, “wt %” is indicated as “%” and “parts by weight” is indicated as “parts”.

### Examples 1-8 and Comparative Examples 1-3

<Essence>

Essences were prepared in accordance with the following method using the components shown in Table 1. The (i) emulsion mean particle diameter, (ii) semitransparency, and (iii) long-term stability of the essences were evaluated using the following method. The results are shown in Table 1.

TABLE 1
(%)

| | | Example | | | | | | | | Comparative Example | | |
|---|---|---|---|---|---|---|---|---|---|---|---|---|
| Component | | 1 | 2 | 3 | 4 | 5 | 6 | 7 | 8 | 1 | 2 | 3 |
| (1) | Ceramide 2 | 0.5 | 0.5 | 0.5 | 0.5 | 0.5 | 0.025 | 2.5 | 0.5 | 0.5 | 0.5 | 0.5 |
| (2) | Isostearic acid | 0.7 | 0.8 | 1 | 1.5 | 2.5 | 0.02 | 2 | 1.4 | 1 | 1 | 1 |
| (3) | Cholesterol | — | 0.2 | 0.5 | 0.5 | — | 0.005 | 0.5 | 0.1 | — | — | — |
| (4) | Polyoxyethylene (60) | 1 | 1 | 1 | 1 | 1 | 0.025 | 2.5 | 1 | 3 | 0.15 | — |
| | hydrogenated castor oil | | | | | | | | | | | |
| (5) | Sodium polyoxyethylene | — | — | — | — | — | — | — | — | — | — | 1 |
| | lauryl ether phosphate | | | | | | | | | | | |
| (6) | Glycerin | — | — | — | — | — | — | 2 | 5 | — | — | — |
| (7) | Diglycerin | — | — | — | — | — | 1 | — | 1 | — | — | — |
| (8) | Alcohol | 7 | 7 | 7 | 7 | 7 | 5 | 5 | 5 | 7 | 7 | 7 |
| (9) | 1,3-Butylene glycol | 10 | 10 | 10 | 10 | 10 | 10 | 10 | 10 | 10 | 10 | 10 |
| (10) | Carboxy vinyl polymer | — | — | — | — | — | 0.1 | — | — | — | — | — |
| (11) | Sodium hydroxide | — | — | — | — | — | 0.03 | — | — | — | — | — |
| (12) | Xanthan gum | — | — | — | — | — | — | 0.1 | — | — | — | — |
| (13) | Native gellan gum | — | — | — | — | — | — | — | 0.05 | — | — | — |
| (14) | Purified water | Balance | Balance | Balance | Balance | Balance | Balance | Balance | Balance | Balance | Balance | Balance |
| Mass ratio of components | | 1.2 | 1.5 | 2 | 2.5 | 3 | 2 | 2 | 2 | 0.5 | 10 | — |
| (a) and (b)/component(c) | | | | | | | | | | | | |
| ([(1) + (2) + (3)]/[(4)]) | | | | | | | | | | | | |
| Evaluation item | | Evaluation result | | | | | | | | | | |
| (i) | Emulsion mean particle | 105 | 156 | 210 | 250 | 298 | 205 | 190 | 202 | 32 | 520 | 430 |
| | diameter (nm) | | | | | | | | | | | |
| (ii) | Semitransparency | 30 | 25 | 5 | 4 | 2 | 6 | 2 | 6 | 60 | 0 | 0 |
| | (600 nm transmissivity (%)) | (*2) | (*2) | (*2) | (*2) | (*2) | (*2) | (*2) | (*2) | (*3) | (*1) | (*1) |
| (iii) | Long-term stability | ∘ | ● | ● | ● | ∘ | ● | ● | ● | x | x | x |

(*1) Opaque
(*2) Semitransparent
(*3) Transparent

(Method of Preparation)
A: Components (1)-(3) were mixed and heated to 70° C.
B: Components (4)-(14) were mixed and heated to 70° C.
C: B was added to A and the mixture was emulsified and cooled to obtain an essence.
   (Method of Evaluation and Judgement)
   (i) Mean Particle Diameter of Emulsion

The emulsion mean particle diameter of the essence was determined using a Sub-micron Particle Analyzer Model N4SD (manufactured by Beckman Coulter, Inc.).

(ii) Semitransparency

Using a UV-2500PC UV-VIS Recording Spectrophotometer (manufactured by Shimadzu Corporation), each of the essences were placed in a 1 cm×1 cm quartz cell and the transmissivity of light at a wavelength of 600 nm was measured.

| (Transmissivity) | (Evaluation) |
|---|---|
| Below 2%: | opaque |
| 2-45%: | semitransparent |
| Greater than 45%: | transparent |

(iii) Long-Term Stability

Each of the essences were stored in a thermostat at 5° C. and 50° C. for one month. The change in appearance (turbidity, separation, and creaming) of each essence compared with the control immediately after preparation was observed by the naked eye to evaluate each essence in accordance with the following four-grade evaluation criteria.

Four-Grade Evaluation Criteria

| (Evaluation) | (Judgment) |
|---|---|
| No change | ● |
| Slight change | ∘ |
| Some degree of change | Δ |
| Considerable degree of change | x |

The above results show that the essences of Examples 1-8 were semitransparent and possessed excellent long-term stability when compared with the essences of Comparative Examples 1-3.

### Example 9

<Face lotion>

| (Component) | (%) |
|---|---|
| 1. Ceramide 2 | 0.5 |
| 2. Isostearic acid | 1.25 |
| 3. Phytosterol | 0.25 |
| 4. Polyoxyethylene (60) hydrogenated castor oil | 1.0 |
| 5. Polyoxyethylene (80) hydrogenated castor oil | 0.02 |
| 6. 1,3-Butylene glycol | 10.0 |
| 7. Glycerin | 3.0 |
| 8. Ethyl alcohol | 5.0 |
| 9. Purified water | Balance |

(Method of Preparation)
A. Components 1-6 were mixed.
B. Components 7-9 were mixed.
C. A was added to B to obtain a face lotion.

The face lotion obtained had an emulsion mean particle diameter of 200 nm, was semitransparent, and possessed excellent long-term stability.

### Example 10

<Essence>

| (Component) | (%) |
|---|---|
| 1. Ceramide 3 | 0.5 |
| 2. Isostearic acid | 1.25 |
| 3. Cholesterol | 0.25 |
| 4. Polyoxyethylene (60) hydrogenated castor oil | 1.0 |
| 5. Polyoxyethylene(20)sorbitan monooleate | 0.02 |
| 6. 1,3-Butylene glycol | 10.0 |
| 7. Glycerin | 10 |
| 8. Polysaccharide produced by alcaligenes | 0.5 |
| 9. Purified water | Balance |

(Method of Preparation)
A. Components 1-6 were mixed.
B. Components 7-9 were mixed.
C. A was added to B to obtain an essence.

The essence obtained above had an emulsion mean particle diameter of 210 nm, was semitransparent, and possessed excellent long-term stability.

### INDUSTRIAL APPLICABILITY

The present invention provides an O/W emulsion semitransparent cosmetic excellent in long-term stability notwithstanding inclusion of ceramide, and having a beautiful appearance and imparting a sense of luxuriousness.

Therefore, the cosmetic of the present invention can make best use of characteristics of ceramides for maintaining moisture in the skin and the like in a wide variety of cosmetics.

Furthermore, since the semitransparent cosmetic of the present invention comprises a smaller amount of surfactants as compared with conventional cosmetics of the same type, the cosmetic excels in safety and feeling during use.

1. An O/W emulsion semitransparent cosmetic comprising the following components (a)-(d):
(a) a ceramide,
(b) an oil component containing a sterol and an isostearic acid,
(c) a nonionic surfactant, and
(d) water,
with a mean particle diameter of 100-300 nm.

2. The semitransparent cosmetic of claim 1, wherein the nonionic surfactant (c) is a polyoxyethylene hydrogenated castor oil.

3. The semitransparent cosmetic of claim 1 or claim 2, wherein the mass ratio of the total amount of the ceramide (a) and the oil component (b) to the amount of the nonionic surfactant (c) is 1.2:1 to 3:1.

4. (canceled)

5. (canceled)

6. The semitransparent cosmetic of any one of claims 1-3, wherein the nonionic surfactant (c) is a polyoxyethylene (60) hydrogenated castor oil.
